(19) United States
(12) Patent Application Publication
(10) Pub. No.: US 2026/0249098 A1
(43) Pub. Date: Aug. 27, 2026

(54) EYE MASK WITH INTEGRATED LIGHTING FOR BILATERAL BRAIN STIMULATION

(71) Applicants: Hunter Atkinson, Mt. Pleasant, UT (US); Nicole Atkinson, Mt. Pleasant, UT (US)

(72) Inventors: Hunter Atkinson, Mt. Pleasant, UT (US); Nicole Atkinson, Mt. Pleasant, UT (US)

(21) Appl. No.: 19/548,224

(22) Filed: Feb. 24, 2026

Related U.S. Application Data
(60) Provisional application No. 63/763,704, filed on Feb. 26, 2025.
(60) Provisional application No. 63/881,012, filed on Sep. 12, 2025.

(51) Int. Cl. A61N 5/06; A61M 21/00; A61M 21/02

(52) U.S. Cl. CPC A61N 5/0618; A61M 21/02; A61M 2021/0027; A61M 2021/0044; A61M 2205/50; A61M 2205/582; A61M 2205/583; A61M 2209/088; A61M 2210/0612; A61M 2210/0618; A61N 2005/0627; A61N 2005/0648; A61N 2005/0652

(57) ABSTRACT

A wearable device includes a wearable support configured to be positioned over eyes of a wearer when worn and a plurality of light-emitting elements coupled to the wearable support. The light-emitting elements are visible to the wearer when the wearable device is worn and extend in a generally linear arrangement that is inferior to, superior to, or both inferior and superior to a forward line of sight of the wearer. The light-emitting elements illuminate sequentially in a direction that extends from a first lateral end portion of the linear arrangement to a second lateral end portion of the linear arrangement that is opposite the first end portion. The wearable device is configured to provide visual stimulation when worn.

### CROSS REFERENCE TO RELATED APPLICATIONS

This application claims the benefit of U.S. Provisional Patent Application Nos. 63/881,012, filed Sep. 12, 2025, and 63/763,704, filed Feb. 26, 2025, which are incorporated by reference herein in their entireties.

### FIELD

This disclosure relates to devices and methods that provide visual stimuli to a wearer to promote a positive resting experience, including relaxation, drowsiness, calmness, and improved cognitive or memory-related states.

### BACKGROUND

Many individuals experience difficulty achieving restful sleep or relaxation due to cognitive arousal, stress, environmental stimuli, or irregular sleep patterns. Devices intended to promote rest or sleep commonly rely on passive techniques, such as light blocking, sound masking, or environmental modification, which may be insufficient for some individuals.

Visual stimulation techniques have been explored in various contexts to influence attention, perception, and eye movement. However, existing approaches to visual stimulation are often limited by rigid placement of light sources, static illumination patterns, overly stimulating visual displays, or configurations that require direct alignment with a forward line of sight. Such approaches may inadvertently promote alertness rather than relaxation, or may be uncomfortable or impractical for use during rest or sleep.

Additionally, many wearable devices that include visual or audio stimulation lack configurability, limiting a wearer's ability to adjust stimulation parameters such as brightness, timing, or pattern. Other devices may rely on complex displays or screens that increase bulk, reduce comfort, or interfere with natural eye positioning. Wearable sleep-related devices that incorporate both visual and audio stimulation are often constrained in design and may not provide coordinated or adjustable stimulation effects.

Accordingly, there is a need for wearable devices and methods that provide visual stimulation in a manner that is comfortable and adjustable. There is further a need for devices capable of delivering controlled visual stimulation patterns positioned relative to a wearer's natural eye line, in some cases in coordination with audio output, to promote relaxation, rest, or sleep-related experiences.

### SUMMARY

Described herein are wearable devices and methods for providing visual stimulation. For example, a wearable device may comprise a wearable support configured to be positioned over eyes of a wearer and a plurality of light-emitting elements coupled to the wearable support. The plurality of light-emitting elements may be arranged in a generally linear configuration relative to a forward line of sight of the wearer and emit light visible to the wearer when the device is worn.

In some examples, the light-emitting elements are positioned inferior to (below), superior to (above), or both inferior and superior to the forward line of sight. The light-emitting elements can be configured to emit light in selectable patterns, such as sequential or scanning illumination. In certain examples, the wearable device may further comprise a controller operable to vary one or more operating parameters. The device may comprise an audio output element configured to provide audible sound that may be output in coordination with emitted light.

Methods of providing visual stimulation are also described, including positioning the wearable device over eyes of a wearer and emitting light from the plurality of light-emitting elements in a pattern visible to the wearer.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a front view of a wearable device, according to an example.

FIG. 2 is a back view of the wearable device of FIG. 1 showing a plurality of light-emitting elements disposed below a natural eye line.

FIG. 3 is a front view of a wearable device, according to another example.

FIG. 4 is a back view of the wearable device of FIG. 3 showing a plurality of light-emitting elements disposed below and above a natural eye line.

FIG. 5 is a front view of a wearable device, according to another example.

FIG. 6 is a back view of the wearable device of FIG. 5 showing a plurality of light-emitting elements configured for vertical displacement.

FIG. 7 is a front view of a wearable device, according to another example.

FIG. 8 is a back view of the wearable device of FIG. 7 showing a plurality of light-emitting elements arranged in a first portion and a second portion spaced apart from the first portion.

FIG. 9 is a perspective view of a wearable device, according to an example.

FIG. 10 is a back view of the wearable device of FIG. 9, illustrating a plurality of light-emitting elements and shaping to contour around facial features.

FIG. 11 is a front view of the wearable device of FIG. 9 showing control features, a charging port, and audio outputs, according to an example.

FIG. 12 illustrates the wearable device of FIG. 10 worn by a wearer and a position of the plurality of light-emitting elements relative to a natural eye line of the wearer.

FIG. 13 is a perspective view of the wearable device of FIG. 10 worn by a wearer.

FIG. 14 illustrates the wearable device of FIG. 10 comprising a portion that is removable.

FIG. 15 is a back view of the wearable device of FIG. 10, according to an example, comprising a liner.

FIG. 16 is a front view of the wearable device of FIG. 15.

FIG. 17 is a top view of a wearable device, according to an example, comprising a controller disposed on a top surface of the wearable device.

FIG. 18 is a schematic showing an electrical layout of the controller of FIG. 17 and a plurality of light-emitting elements.

FIG. 19 is a back view of the wearable device of FIG. 17 illustrating the controller coupled to the wearable device via wiring.

FIG. 20 is a front view of the wearable device of FIG. 17.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved. The scope of this disclosure includes any features disclosed herein combined with any other features disclosed herein, unless physically impossible.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

As used in this disclosure and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In the description, certain terms may be used such as “forward,” “front,” “rear,” “back,” “up,” “down,” “upper,” “lower,” “horizontal,” “vertical,” “left,” “right,” “longitudinal,” “lateral,” and the like. These terms are used, where applicable, to provide some clarity of description when dealing with relative relationships. However, these terms are not intended to imply absolute relationships, positions, and/or orientations. For example, with respect to an object, an “upper” surface can become a “lower” surface by turning the object over. Nevertheless, it is still the same object.

Similar components in different embodiments are described in the specification and illustrated in the figures with similar reference numbers for improved understanding and readability. However, it should be understood that this numbering convention is merely for convenience and is not intended to limit and/or exclude any claim scope.

Although there are alternatives for various components, parameters, operating conditions, etc., set forth herein, that does not mean that those alternatives are necessarily equivalent and/or perform equally well. Nor does it mean that the alternatives are listed in a preferred order unless stated otherwise.

As used herein, “e.g.” means “for example,” and “i.e.” means “that is.”

### Overview of the Disclosed Technology

The disclosed technology relates to wearable systems and methods for delivering controlled visual stimulation to a wearer. The technology is directed to positioning light-emitting elements within a wearable structure. By arranging the light-emitting elements in a generally linear configuration relative to the wearer's natural eye line, the disclosed technology enables visual stimulation that may be experienced comfortably during rest or relaxation.

In various examples, the wearable structure supports one or more light-emitting elements configured to emit light in selectable patterns, including sequential or scanning patterns that extend laterally across a visual field. At least the position, pattern, direction, rate, and brightness of emitted light may be adjustable, allowing the technology to accommodate different usage scenarios and preferences.

In some examples, the disclosed technology may further integrate audio output, enabling coordinated or independent delivery of audible sound in combination with visual stimulation. Control features may allow a wearer to select operating parameters, including timing, intensity, and pattern characteristics of the light output.

The disclosed technology may be implemented in various wearable form factors, including headsets, sleep masks, or eye covers, and may incorporate comfort features, alignment indicators, and light-shielding elements. The technology is adaptable to different configurations and use cases and is not limited to the specific examples described herein.

### Examples of the Disclosed Technology

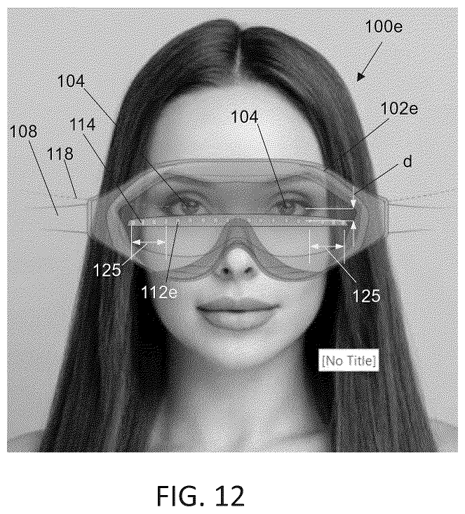

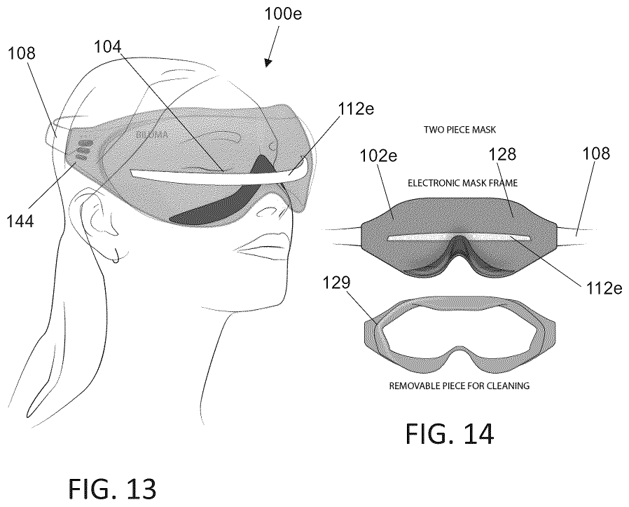

A wearable device 100 (e.g., wearable devices disclosed herein, such as 100a, 100b, 100c, 100d, 100e, 100f) comprises a wearable support 102 configured to be positioned over eyes 104 of a wearer when the wearable device 100 is worn (see FIGS. 12-13). The wearable support 102 (e.g., wearable supports disclosed herein, such as 102a, 102b, 102c, 102d, 102e, 102f) may be formed of comfortable, lightweight materials and may be configured to wrap around the eyes and/or head of the wearer. In some examples, the material of the wearable support is configured to block, attenuate, or otherwise inhibit ambient light from entering a space between the wearable support and the wearer's eyes. Non-limiting examples of the wearable support 102 include a headset, sleep mask, eye cover, visor, or other face-worn structure. The wearable support 102 may include retention elements 108 such as elastic bands, straps, fasteners, or the like to maintain the device in position during use.

Coupled to the wearable support is a light source, such as a plurality of light-emitting elements 112 (e.g., the plurality of light-emitting elements disclosed herein such as 112a, 112b, 112c, 112d, 112e, 112f) that is configured to emit light visible to the wearer when the wearable device is worn. Each light-emitting element 114 of the plurality of light-emitting elements 112 (e.g., the plurality of light-emitting elements disclosed herein such as 112a, 112b, 112c, 112d, 112e, 112f) can be a light-emitting diode (LED) or other suitable light element. For example, the LEDs may be arranged as an LED light strip. Any of the plurality of light-emitting elements 112 disclosed herein may emit light in one or more wavelengths. In some examples, the plurality of light-emitting elements 112 may emit yellow or non-blue light wavelengths to reduce stimulation of wakefulness centers in the brain.

Any plurality of light-emitting elements disclosed herein may be coupled to a wearable support by any suitable attachment technique, including being embedded within the wearable support, mounted on an inner surface of the wearable support, secured within a pocket or channel formed in the wearable support, or attached using adhesive, fasteners, stitching, or other mechanical or chemical coupling mechanisms.

In some examples, the plurality of light-emitting elements 112 can extend in a generally linear arrangement that is inferior to (also referred to herein as “below”) a forward line of sight (also referred to herein as “natural eye line”) 118 of the wearer when the wearable device 100 is worn. The generally linear arrangement may be superior to (also referred to herein as “above”) the forward line of sight 118 of the wearer when the wearable device 100 is worn. In some examples, the generally linear arrangement may include linear arrangements that are both inferior and superior to the forward line of sight 118 of the wearer when the wearable device 100 is worn. As used herein, the term linear refers to an arrangement extending along a generally continuous path, which may be straight or curved, and is not limited to strict geometric straightness. The linear arrangement further denotes that the light-emitting elements are positioned in a deliberate, ordered progression along the path.

In some examples, the light-emitting elements 112 can be arranged a distance d, d′ that is approximately 2-10 mm, or within 5 mm, inferior or superior to the forward line of sight 118. This arrangement of light-emitting elements 112 may draw the wearer's gaze downward or upward relative to a natural straight-ahead gaze. That is, an inferior arrangement may draw the wearer's gaze downward and a superior arrangement may draw the wearer's gaze upward as will be described further below.

In some examples, the light-emitting elements can be generally level or aligned with the forward line of sight 118.

As used herein, the forward line of sight 118 refers to a direction extending generally straight ahead from the eyes 104 of a wearer when the eyes are in a natural, relaxed viewing position. The forward line of sight 118 may correspond to a natural resting eye line of the wearer. As used herein, an interpupillary line 119 refers to a line extending between the pupils of the eyes 104 when the eyes are in a natural, relaxed viewing position.

Positioning light-emitting elements inferior to the natural eye line 118 may encourage a downward gaze and reduced cognitive arousal, which may counter wakefulness centers and promote relaxation and drowsiness. Such visual stimulation may be beneficial, for example, at bedtime or during periods of rest. In some examples, the light-emitting elements may be positioned superior to the eye line 118 to promote alertness or wakefulness.

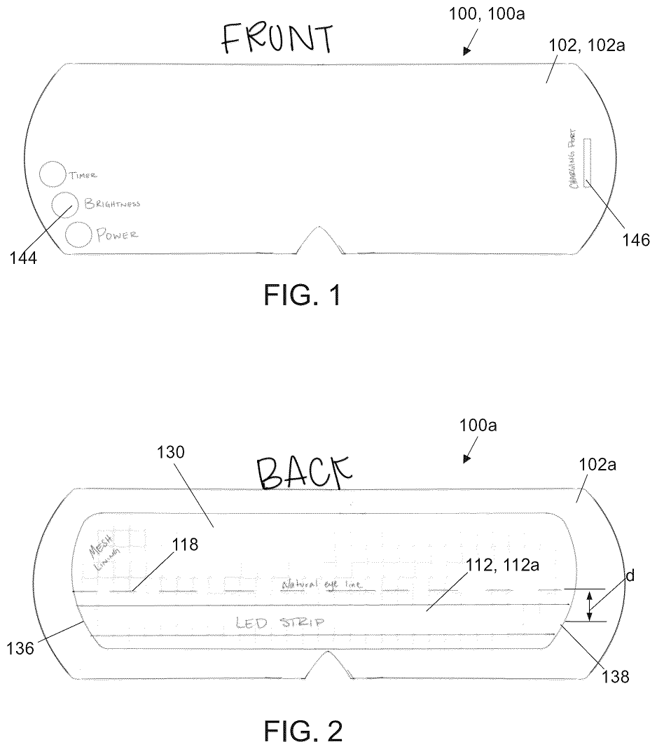

FIGS. 1-2 illustrate a wearable device 100a, according to an example, comprising a wearable support 102a and a plurality of light-emitting elements 112a coupled to the wearable support 102a. The light-emitting elements 112a extend in a generally linear arrangement that is inferior to the forward line of sight 118 of the wearer by the distance d when the wearable device 100a is worn.

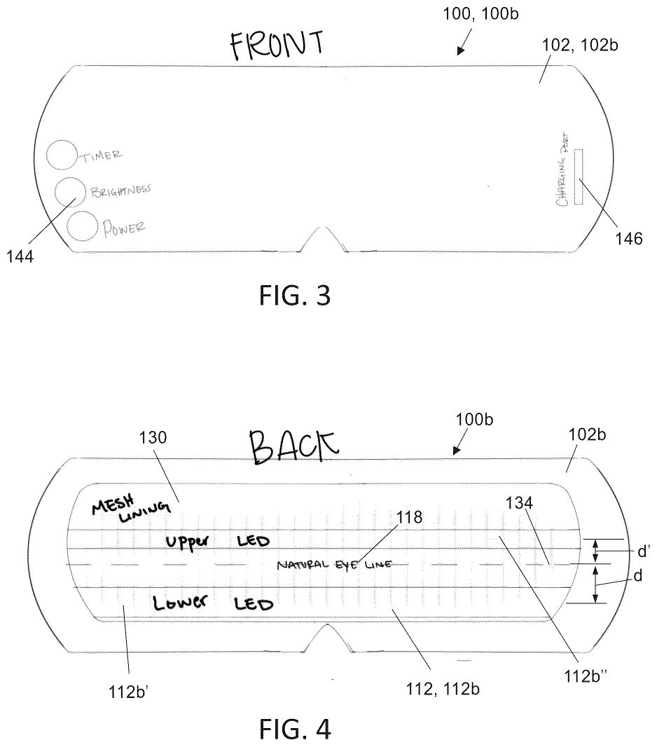

FIGS. 3-4 illustrate a wearable device 100b, according to another example, comprising a wearable support 102b and a plurality of light-emitting elements 112b coupled to the wearable support 102b. The light-emitting elements 112b extend in a first generally linear arrangement 112b′ that is inferior to the forward line of sight 118 of the wearer by the distance d and a second generally linear arrangement 112b″ that is superior to the forward line of sight 118 by a distance d′ when the wearable device 100b is worn. In some examples, the distances d, d′ can be the same. In some examples, the distances d, d′ can be different.

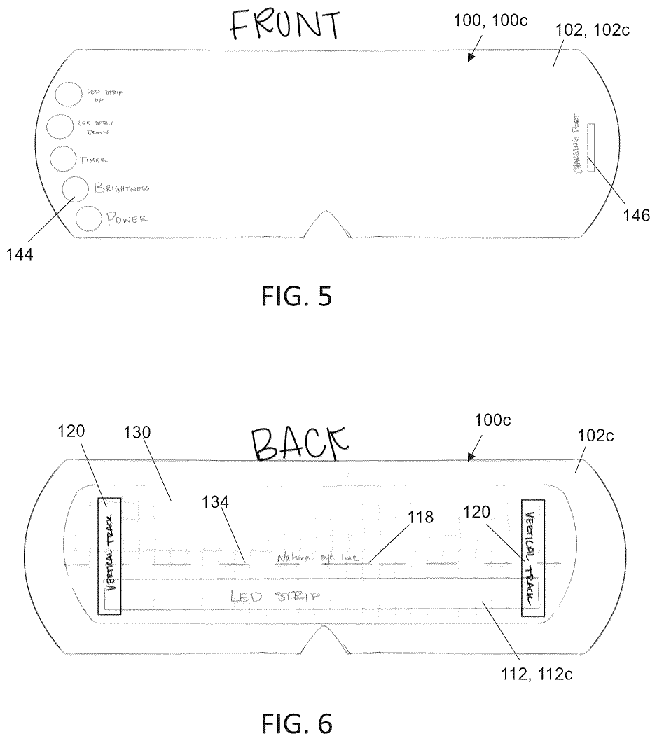

In some examples, light-emitting elements can be configured for vertical displacement relative to a wearable support. FIGS. 5-6 illustrate a wearable device 100c, according to an example, comprising a wearable support 102c and a plurality of light-emitting elements 112c coupled to the wearable support 102c for vertical displacement relative thereto. The plurality of light-emitting elements 112c can be mounted to the wearable support 102c via one or more vertical tracks 120 or other adjustment mechanism(s) that allow the linear arrangement to be displaced between different vertical positions. This adjustment allows the wearer to control whether the light-emitting elements are positioned inferior, superior, or closer to the natural eye line.

In some examples, the vertical adjustment may be achieved using hook-and-loop fasteners, buttons, snaps, or any other mechanism configured to permit adjustable positioning relative to the mask. Accordingly, the plurality of light-emitting elements 112c may be mounted to the wearable support 102c via one or more positioning elements that allow adjustment of the relative position of the light-emitting elements.

The generally linear arrangement of light-emitting elements may be straight, substantially straight, segmented, curved, or arched. In some examples, the linear arrangement can be substantially parallel to the interpupillary line 119 of the wearer. In other examples, the linear arrangement can be arched, with an apex disposed between the eyes of the wearer, such as above a nose region. Such an arched configuration may reduce or prevent occlusion of one or more light-emitting elements by a nose portion of the wearable device or the wearer's nose. As a result, when the light-emitting elements are illuminated sequentially, the continuity of the perceived illumination pattern may be maintained without visual interruption caused by obscured light-emitting elements.

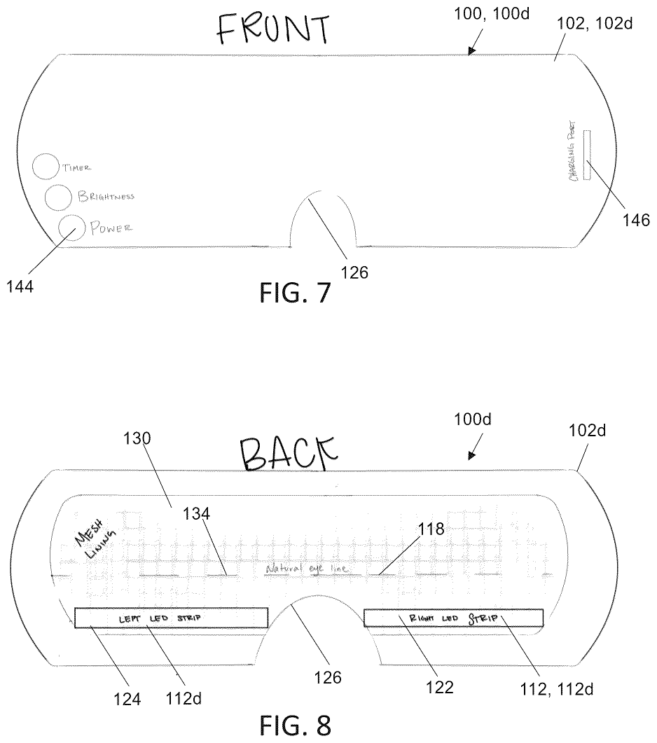

In some examples, the linear arrangement of light-emitting elements can comprise two or more separate portions. For example, the linear arrangement can comprise a first portion adjacent a first eye of the wearer and a second portion adjacent a second eye of the wearer. FIGS. 7-8 illustrate a wearable device 100d, according to an example, comprising a wearable support 102d and a plurality of light-emitting elements 112d coupled to the wearable support 102d. The plurality of light-emitting elements 112d are arranged with a first portion 122 disposed adjacent or below a first eye and a second portion 124 disposed adjacent or below a second eye. This configuration may be particularly useful when the wearable support leaves the wearer's nose uncovered via a nose slot 126. The first and second portions 122, 124 may be configured such that they extend laterally outward on their respective sides of the nose from substantially adjacent the nose or the nose slot 126.

Each portion 122, 124 may include a respective subset of the plurality of light-emitting elements 112d that work in concert to provide a scanning visual stimulus across a face. That is, the plurality of light-emitting elements 112d can travel sequentially from an outside or lateral end portion of the first eye to an outside, lateral end portion of the second eye. In some examples, the portions 122, 124 may be disposed above the eyes.

In some examples, the first and second portions 122, 124 can each form shorter segments than the segments depicted in FIG. 8. Such shorter segments may be disposed laterally from the nose slot 126, for example, adjacent outer corners of the eyes. That is, the first and second portions 122, 124 can each be spaced apart laterally a greater distance from the nose or nose slot 126 than the spacing shown in FIG. 8. The first and second portions 122, 124 may extend on either side horizontally (e.g., laterally) a distance 125 outside the user's natural resting eye position from one or both eyes (see FIG. 12 for 125 relative to the eyes of the wearer). For example, this distance may be approximately 1-3 centimeters. In some examples, the first and second portions 122, 124 may be illuminated alternately, which may produce a similar bilateral stimulation effect.

In some examples, the linear arrangement can extend across substantially the entire visible area inside the wearable support. The linear arrangement, in some instances, can extend laterally beyond the natural resting eye position by the distance 125 as shown in FIG. 12. The distance 125 may be approximately 1-3 centimeters on one or both sides of the eyes.

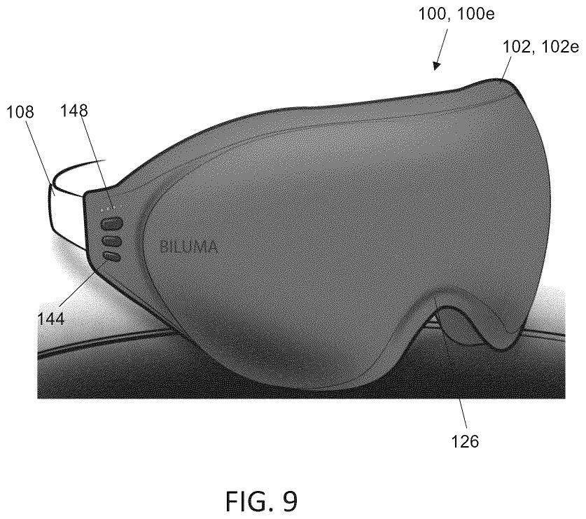

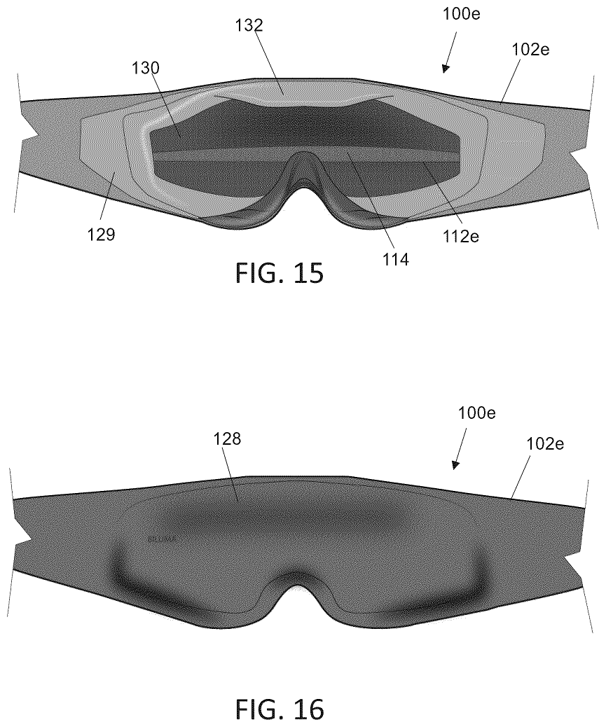

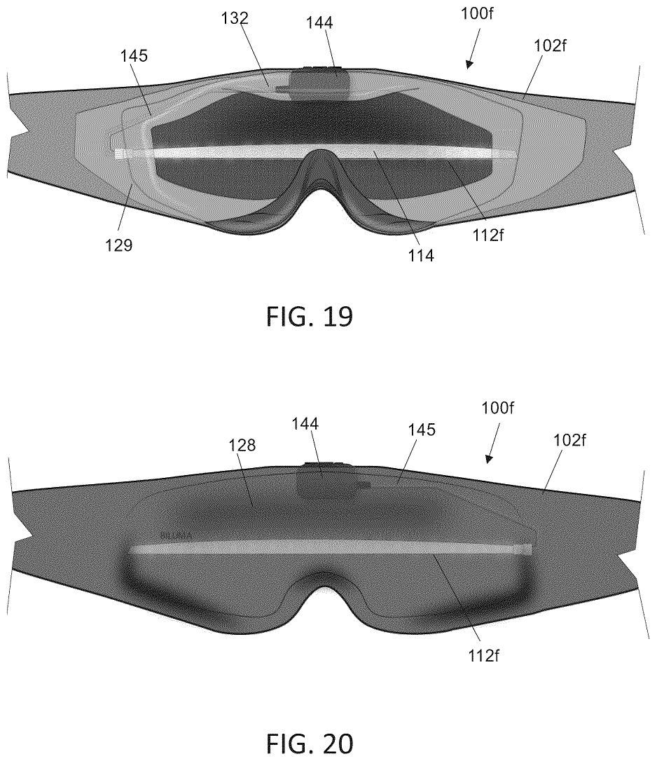

In some examples, the wearable support 102 can be configured to leave at least a portion of the wearer's nose 110 uncovered, thereby improving comfort and fit. That is, the wearable support 102 may be shaped to contour around facial features while maintaining a consistent position relative to the wearer's eyes. FIGS. 9-17 and 19-20 illustrate wearable devices 100e, 100f, according to an example, comprising wearable supports 102e, 102f having such shaping and contouring.

In some examples, the wearable supports 102e, 102f may comprise a frame 128 and a removable member 129 coupled to the frame 128. The removable member 129 may be configured to contact a face of a wearer when the wearable device is worn and may be selectively detached from the frame 128 for cleaning, replacement, or interchange with another removable member. In some examples, the removable member 129 is formed of a fabric, foam, or other compliant material and is removably coupled to the frame 128 using fasteners, adhesive, magnetic elements, or similar attachment mechanisms.

It is appreciated that contouring and shaping depicted for the wearable supports 102e, 102f shown in FIGS. 9-17 and 19-20 may be applied to any wearable support described herein (e.g., wearable supports 102a, 102b, 102c, 102d). Moreover, any wearable support described herein may comprise a removable member as described above.

In some examples, the wearable support 102 (e.g., any wearable supports 102a, 102b, 102c, 102d, 102e, 102f disclosed herein) can include one or more liners 130 disposed over the plurality of light-emitting elements. The one or more liners 130 may be formed of a lightweight mesh-like, translucent, or diffusive material configured to dim or soften direct exposure to the visual stimulus. This diffusion may help avoid alerting wakefulness centers of the brain while maintaining perceptibility.

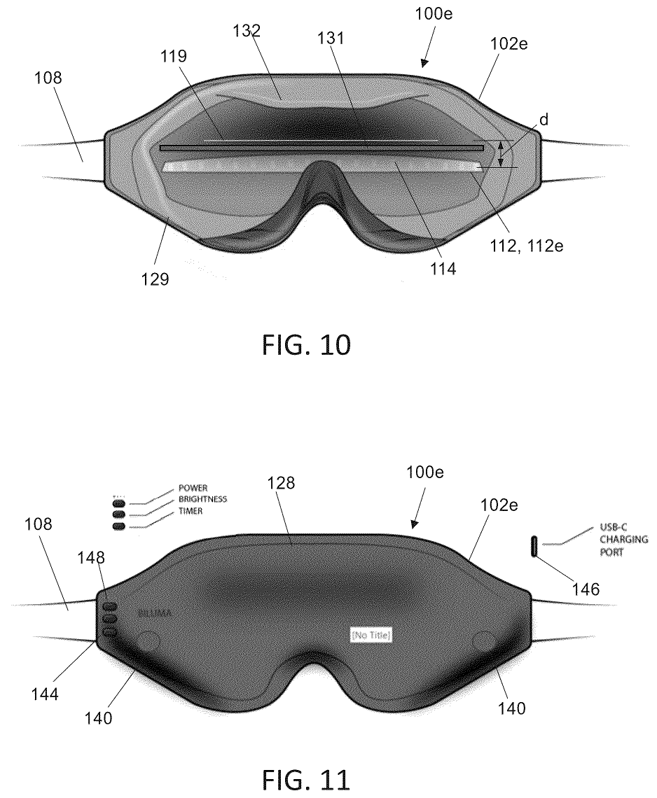

The wearable support 102 (e.g., any of the wearable supports 102a, 102b, 102c, 102d, 102e, 102f disclosed herein) may comprise one or more features configured to deflect light downward and/or deter light emitted from the plurality of light-emitting elements 112 from extending above the line of sight 118. In some examples, a cloth, opaque, or light-blocking material may extend from an inner surface of the wearable support toward the wearer's eyes above the natural eye line to form a visor-like structure (also referred to herein as a “visor”) 131 (FIG. 10). The visor 131 may reduce the likelihood that the wearer perceives light emitted above the eye line. Additionally or alternatively, the plurality of light-emitting elements 112 may be angled downward such that emitted light is directed away from upward reflection.

In some examples, the wearable support 102 (e.g., any wearable supports 102a, 102b, 102c, 102d, 102e, 102f disclosed herein) may comprise a shield 132 configured to block or reduce external light from reaching the wearer's eyes, thereby enhancing the effectiveness of the visual stimulation.

The wearable support 102 (e.g., any wearable supports 102a, 102b, 102c, 102d, 102e, 102f disclosed herein) may include one or more indicators configured to assist alignment of the wearable device over the eyes of the wearer. Such indicators may include visual markings, stitching, seams, tactile features, or alignment guides. For example, a horizontal stitch 134 may be positioned at the natural eye line to assist placement of the light-emitting elements below the eye line.

The wearable support 102 (e.g., any wearable supports 102a, 102b, 102c, 102d, 102e, 102f disclosed herein) may be configured for use with a nose breathing strip. For example, a nose breathing strip having one or more coupling elements can be arranged on and attached to one or both sides of the wearer's nose. The one or more coupling elements on the nose breathing strip can correspond to and connect with one or more coupling elements on the wearable support 102. For example, the coupling elements can be magnetic (e.g., one or more magnets or a magnetic strip). When the magnetic elements on the nose breathing strip and the magnetic elements on the wearable support 102 are brought into proximity with each other (e.g., when the wearable support 102 is positioned on a wearer who is wearing the nose breathing strip), magnetic attraction between the magnets pulls the nasal sidewalls of the wearer outward, thereby widening the nasal passages and increasing airflow through the nose.

In some examples as introduced above, the light-emitting elements of the plurality of light-emitting elements 112 (e.g., the plurality of light-emitting elements disclosed herein such as 112a, 112b, 112c, 112d, 112e, 112f) can be configured to illuminate sequentially in a direction extending from a first, lateral end portion of a linear arrangement to a second, lateral end portion of the linear arrangement opposite the first lateral end portion. By way of example, the plurality of light-emitting elements 112a shown in FIG. 2 can illuminate sequentially from a first end portion 136 to a second end portion 138 of the linear arrangement, or vice versa. This sequential illumination may create a scanning visual stimulus that moves left-to-right (e.g., generally horizontally), right-to-left, or in alternating directions. For example, the plurality of light-emitting elements can be configured to illuminate sequentially such that successive light-emitting elements are activated one after another to create a perceived moving illumination pattern.

This scanning visual stimulus may induce eye movements that replicate or approximate eye movements present during REM sleep stages. Such eye movements may provide benefits including relaxation, drowsiness, calmness, clear thinking, and appropriate memory processing.

The rate of sequential illumination (e.g., scanning) may be fixed. In some examples, the rate of sequential illumination may be variable. In some examples, the scanning speed can begin at a predetermined rate and gradually decrease over time, allowing eye-tracking movements to slow naturally as the wearer drifts into sleep.

In some examples, the scanning speed can remain substantially constant throughout operation or increase.

In some instances, the plurality of light-emitting elements 112 (e.g., the plurality of light-emitting elements disclosed herein such as 112a, 112b, 112c, 112d, 112e, 112f) can turn off after a duration of time.

The direction of sequential illumination or scanning may also be varied during operation, including reversing direction or alternating between directions.

The brightness of the light-emitting elements 112 (e.g., the plurality of light-emitting elements disclosed herein such as 112a, 112b, 112c, 112d, 112e, 112f) may be selectively varied. In some examples, brightness is adjustable to allow the visual stimulus to be perceived with eyes open or closed. For example, increased brightness may allow the visual stimulus to be perceived through closed eyelids.

In certain examples, the wearable device includes a plurality of light-emitting elements positioned superior to the natural eye line 118 as shown in FIG. 4 (e.g., the second generally linear arrangement 112b″). This second generally linear arrangement 112b″ may be activated after the first generally linear arrangement 112b′ is activated. That is, in some examples, upper visual stimulation may be activated after the lower visual stimulation is deactivated. In some examples, this upper visual stimulation may be activated by a timer to promote alertness or wakefulness after rest. This upper visual stimulation may emit scanning light. In some instances, this upper visual stimulation may comprise constant or non-scanning light.

In some examples, any wearable device 100 disclosed herein (e.g., the wearable devices 100a, 100b, 100c, 100d, 100e, 100f) may further comprise one or more audio output elements 140 configured to provide audible sound to the wearer (see for example FIG. 11). The one or more audio output elements 140 may include speakers positioned near or over the wearer's ears or may include bone-conduction elements.

In some examples, the one or more audio output elements 140 are configured to connect wirelessly, such as via Bluetooth, to an external device (e.g., a mobile device such as a smartphone, tablet, computer, etc.), allowing the wearer to select audio content. In other examples, the one or more audio output elements 140 may emit preprogrammed sounds, such as white noise or tones at specific frequencies associated with relaxation. For example, frequencies in a range of approximately 400-450 Hz (e.g., 432 Hz) are often described as more natural and harmonious, promoting relaxation and stress reduction. Frequencies in a range of approximately 500-550 Hz (e.g., 528 Hz) are known as the “love frequency” which can be linked to emotional healing and calmness. Such frequencies of sound may be emitted by the one or more audio output elements 140.

In some examples, audio output can alternate between ears in coordination with the scanning visual stimulus, creating a bilateral stimulation effect. In other examples, audio output can be provided simultaneously or constant to both ears.

Any wearable device disclosed herein (e.g., the wearable devices 100a, 100b, 100c, 100d, 100e, 100f) may further comprise a controller 144 operable by the wearer to select one or more operating settings. The controller 144 may be connected to the light-emitting elements and/or audio output elements via wired or wireless connection. The controller 144 may comprise user controls such as buttons, switches, or other suitable actuators that allow user control of activation or power, timers, brightness, scanning speed, scanning direction, duration of operation, or other operation parameters. Such user controls may be disposed on an exterior portion of the wearable support and be easily accessible. Additional user controls may allow the wearer to move the light-emitting elements upward or downward within the visual space of the wearable support, as described above in connection with FIG. 6.

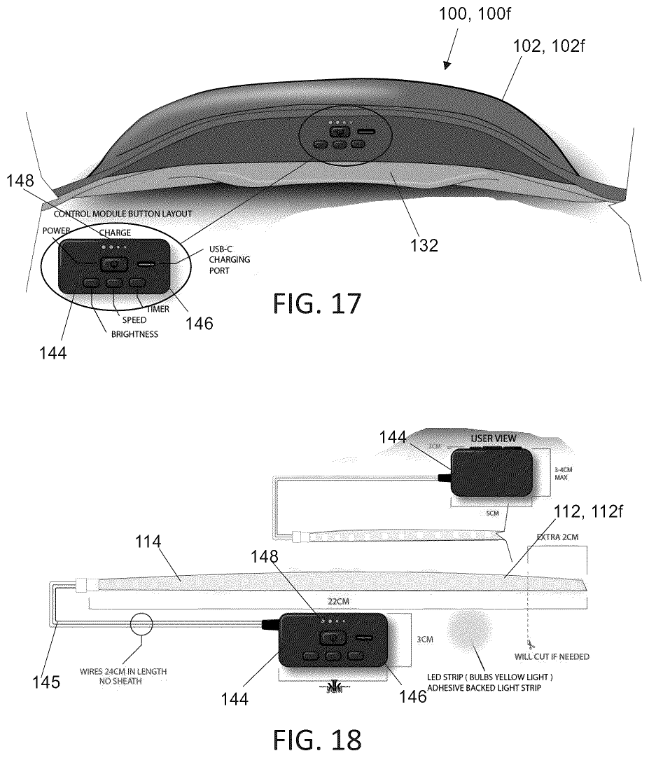

FIGS. 17, 19, and 20 illustrate a wearable device 100f, according to an example, comprising a wearable support 102f and a plurality of light-emitting elements 112f coupled to the wearable support 102f via a wired connection 145. The controller 144 is shown arranged on an upper surface of the wearable support 102f. In some examples, as depicted in FIG. 9, the controller 144 may be disposed on a lateral surface of a wearable support, although any user-accessible placement is possible.

Any of the wearable devices disclosed herein can be configured to connect wirelessly, such as via Bluetooth, to an external device (e.g., a mobile device such as a smartphone, tablet, computer, etc.) having a program or app that allows the wearer to customize and/or control their experience, for example, before or during use. For example, via the program or app on the external device, the wearer may select the brightness, scanning speed, scanning direction, duration of operation, selectable pattern, and/or other operating parameters. The wearer may select one or more initial operating parameters (e.g., speed, brightness, pattern, color, etc.) and may adjust one or more operating parameters during use. In some examples, using the program or app, the user may be able to set a wake-up alarm that triggers the lights to turn on at a time specified by the user.

Any of the wearable devices disclosed herein can incorporate sleep tracking technology, for example sensors, that can monitor, record, and/or analyze sleep metrics of the wearer. For example, the sleep tracking technology may inform the wearer of sleep stages, sleep duration, sleep quality and/or sleeping patterns. Sleep sensors can be configured to detect physiological and/or motion-related data, such as heart rate, temperature, body movement, and/or sleep position of the wearer. Any combination of one or more such sensors can be arranged on a wearable device (e.g., the wearable devices 100a, 100b, 100c, 100d, 100e, 100f) to provide sleep data. In some examples, sleep sensors may comprise an accelerometer that measures the movement of the wearer during sleep, a thermometer that measures the temperature of the wearer during sleep, and/or a pulse sensor that measures the wearer's pulse. In some examples, a thermometer and/or pulse sensor may be arranged on a wearable device, for instance, in position over or near the wearer's temple. Additionally or alternatively, an accelerometer may be arranged on a wearable device, for instance, near the front middle of the device (e.g., between the wearer's eyes or above the nose).

In some examples, any wearable device disclosed herein can be rechargeable and include a lightweight battery fastened to or housed within the wearable support. The wearable device may comprise a charging port 146 and a battery fill indicator 148.

Methods described herein include positioning the wearable device over eyes of a wearer and emitting light from the plurality of light-emitting elements in a pattern visible to the wearer. The method may include selectively varying the pattern, brightness, rate, or direction of light emission, including sequential illumination. The method may be performed prior to sleep, after waking, or at any time the wearer desires the described benefits.

According to an example, wearable devices disclosed herein can comprise any combination of control areas, features, and/or settings detailed above. The examples described herein are illustrative and not limiting. Features described in connection with one example may be combined with features of other examples unless expressly stated otherwise. Relative terms such as “inferior,” “superior,” “lateral,” and “forward” are used for descriptive purposes and refer to the orientation of the wearable device when worn.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

The features described herein with regard to any example can be combined with other features described in any one or more of the other examples, unless otherwise stated. For example, any one or more features of one wearable device can be combined with any one or more features of another wearable device.

Example 1. A wearable device comprising: a wearable support configured to be positioned over eyes of a wearer when the wearable device is worn; and a plurality of light-emitting elements coupled to the wearable support and visible to the wearer when the wearable device is worn, wherein the plurality of light-emitting elements extends in a generally linear arrangement that is inferior, superior, or both inferior and superior to a forward line of sight of the wearer when the wearable device is worn, and wherein light-emitting elements of the plurality of light-emitting elements are configured to illuminate sequentially in a direction that extends from a first lateral end portion of the linear arrangement to a second lateral end portion of the linear arrangement that is opposite the first lateral end portion.

Example 2. The device of any example herein, particularly example 1, wherein the light-emitting elements are arranged only inferior to the forward line of sight of the wearer when the wearable device is worn.

Example 3. The device of any example herein, particularly example 1, wherein a rate at which the light-emitting elements illuminate is configured to vary.

Example 4. The device of any example herein, particularly example 1, wherein the direction that the light-emitting elements illuminate changes.

Example 5. The device of any example herein, particularly example 1, wherein the linear arrangement comprises a first portion of the plurality of light-emitting elements and a second portion of the plurality of light-emitting elements disposed a distance from the first portion, wherein the first portion is adjacent a first eye of the wearer and the second portion is adjacent a second eye of the wearer.

Example 6. The device of any example herein, particularly example 5, wherein the first and second portions of the plurality of light-emitting elements are arranged on different sides of the wearer's nose from one another.

Example 7. The device of any example herein, particularly example 1, wherein the linear arrangement is substantially parallel to an interpupillary line of the wearer.

Example 8. The device of any example herein, particularly example 1, wherein the wearable support comprises a first side configured to face the eyes of the wearer when worn and a second side opposite the first side, wherein a liner is coupled to the first side of the wearable support and disposed over the plurality of light-emitting elements.

Example 9. The device of any example herein, particularly example 1, wherein the wearable support comprises an indicator configured to assist alignment of the wearable device over the eyes of the wearer.

Example 10. The device of any example herein, particularly example 1, further comprising an audio output element configured to provide audible sound to the wearer.

Example 11. The device of any example herein, particularly example 1, wherein the plurality of light-emitting elements is arranged 2-10 mm inferior or superior to the forward line of sight of the wearer when the wearable device is worn.

Example 12. The device of any example herein, particularly example 1, wherein the plurality of light-emitting elements are arranged lateral one or both eyes of the wearer.

Example 13. The device of any example herein, particularly example 1, wherein the wearable support comprises a shield configured to block external light from the eyes of the wearer when the wearable device is worn.

Example 14. The device of any example herein, particularly example 1, further comprising a controller operable by the wearer to select one or more operating settings.

Example 15. The device of any example herein, particularly example 1, wherein the plurality of light-emitting elements is configured to be displaced between a first position and a second position that is inferior or superior relative to the first position.

Example 16. A method of providing a visual stimulus, the method comprising: positioning a wearable device over eyes of a wearer, wherein the wearable device comprises: a wearable support; and a plurality of light-emitting elements disposed on the wearable support and extending in a generally linear arrangement that is inferior, superior, or both inferior and superior to a forward line of sight of the wearer when the wearable device is worn, and emitting light from the plurality of light-emitting elements in a pattern that is visible to the wearer, wherein the pattern comprises sequentially illuminating light-emitting elements of the plurality of light-emitting elements in a direction that extends from a first lateral end portion of the linear arrangement to a second lateral end portion of the linear arrangement that is opposite the first end portion.

Example 17. The method of any example herein, particularly example 16, further comprising varying the direction of sequential illumination.

Example 18. The method of any example herein, particularly example 16, further comprising varying a rate of sequential illumination.

Example 19. The method of any example herein, particularly example 16, further comprising varying a brightness of the plurality of light-emitting elements.

Example 20. The method of any example herein, particularly example 16, wherein the light-emitting elements are arranged only inferior to the forward line of sight of the wearer when the wearable device is worn.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A wearable device comprising:
a wearable support configured to be positioned over eyes of a wearer when the wearable device is worn; and
a plurality of light-emitting elements coupled to the wearable support and visible to the wearer when the wearable device is worn,
wherein the plurality of light-emitting elements extends in a generally linear arrangement that is inferior, superior, or both inferior and superior to a forward line of sight of the wearer when the wearable device is worn, and
wherein light-emitting elements of the plurality of light-emitting elements are configured to illuminate sequentially in a direction that extends from a first lateral end portion of the linear arrangement to a second lateral end portion of the linear arrangement that is opposite the first lateral end portion.

2. The device of claim 1, wherein the light-emitting elements are arranged only inferior to the forward line of sight of the wearer when the wearable device is worn.

3. The device of claim 1, wherein a rate at which the light-emitting elements illuminate is configured to vary.

4. The device of claim 1, wherein the direction that the light-emitting elements illuminate changes.

5. The device of claim 1, wherein the linear arrangement comprises a first portion of the plurality of light-emitting elements and a second portion of the plurality of light-emitting elements disposed a distance from the first portion, wherein the first portion is adjacent a first eye of the wearer and the second portion is adjacent a second eye of the wearer.

6. The device of claim 5, wherein the first and second portions of the plurality of light-emitting elements are arranged on different sides of the wearer's nose from one another.

7. The device of claim 1, wherein the linear arrangement is substantially parallel to an interpupillary line of the wearer.

8. The device of claim 1, wherein the wearable support comprises a first side configured to face the eyes of the wearer when worn and a second side opposite the first side, wherein a liner is coupled to the first side of the wearable support and disposed over the plurality of light-emitting elements.

9. The device of claim 1, wherein the wearable support comprises an indicator configured to assist alignment of the wearable device over the eyes of the wearer.

10. The device of claim 1, further comprising an audio output element configured to provide audible sound to the wearer.

11. The device of claim 1, wherein the plurality of light-emitting elements is arranged 2-10 mm inferior or superior to the forward line of sight of the wearer when the wearable device is worn.

12. The device of claim 1, wherein the plurality of light-emitting elements are arranged lateral one or both eyes of the wearer.

13. The device of claim 1, wherein the wearable support comprises a shield configured to block external light from the eyes of the wearer when the wearable device is worn.

14. The device of claim 1, further comprising a controller operable by the wearer to select one or more operating settings.

15. The device of claim 1, wherein the plurality of light-emitting elements is configured to be displaced between a first position and a second position that is inferior or superior relative to the first position.

16. A method of providing a visual stimulus, the method comprising:
positioning a wearable device over eyes of a wearer, wherein the wearable device comprises:
a wearable support; and
a plurality of light-emitting elements disposed on the wearable support and extending in a generally linear arrangement that is inferior, superior, or both inferior and superior to a forward line of sight of the wearer when the wearable device is worn, and
emitting light from the plurality of light-emitting elements in a pattern that is visible to the wearer,
wherein the pattern comprises sequentially illuminating light-emitting elements of the plurality of light-emitting elements in a direction that extends from a first lateral end portion of the linear arrangement to a second lateral end portion of the linear arrangement that is opposite the first end portion.

17. The method of claim 16, further comprising varying the direction of sequential illumination.

18. The method of claim 16, further comprising varying a rate of sequential illumination.

19. The method of claim 16, further comprising varying a brightness of the plurality of light-emitting elements.

20. The method of claim 16, wherein the light-emitting elements are arranged only inferior to the forward line of sight of the wearer when the wearable device is worn.
